(19) United States
(12) Patent Application Publication
(10) Pub. No.: US 2003/0068321 A1
(43) Pub. Date: Apr. 10, 2003

(54) Methods of effecting neuronal activity

(75) Inventors: Gregory S. Hamilton, Catonsville, MD (US); Ling Wei, Lutherville, MD (US); Joseph P. Steiner, Mt. Airy, MD (US)

Correspondence Address: HOWREY SIMON ARNOLD & WHITE LLP, /GUILFORD PHARMACEUTICALS, 1299 PENNSYLVANIA AVENUE, N.W., WASHINGTON, DC, 20004, US

(73) Assignee: Guilford Pharmaceuticals, Inc.

(21) Appl. No.: 09/947,700

(22) Filed: Sep. 7, 2001

(51) Int. Cl. A61K 38/48; A61K 39/395

(52) U.S. Cl. 424/146.1; 424/94.63

(57) ABSTRACT

This invention is related to methods of effecting neuronal activity with non-immunosuppressive neurotrophic low molecular weight, small molecule cyclophilin inhibitor compounds having an affinity for cyclophilin-type immunophilins. The methods of this invention are useful in the stimulation of damaged neurons, promotion of neuronal regeneration, prevention of neurodegeneration, and treatment of neurological disorders.

This invention is related to methods of effecting neuronal activity with non-immunosuppressive neurotrophic low molecular weight, small molecule cyclophilin inhibitor compounds having an affinity for cyclophilin-type immunophilins.

The term immunophilin refers to a number of proteins that serve as receptors for the principal immunosuppressant drugs, cyclosporin A (CsA), FK506 and rapamycin. Known classes of immunophilins are cyclophilins and FK506 binding proteins, or FKBPs. Cyclosporin A binds to cyclophilin A while FK506 and rapamycin bind to FKBP12. These immunophilin-drug complexes interface with various intracellular signal transduction systems, especially the immune and nervous systems.

Immunophilins are known to have peptidyl-prolyl isomerase (PPIase), or rotamase, enzyme activity. It has been determined that rotamase enzyme activity plays a role in the catalyzation of the interconversion of the cis and trans isomers of peptide and protein substrates for the immunophilin proteins.

Immunophilins were originally discovered and studied in the immune tissue. It was initially postulated by those skilled in the art that inhibition of the immunophilins' rotamase activity leads to inhibition of T-cell proliferation, thereby causing the immunosuppressive activity exhibited by immunosuppressant drugs, such as cyclosporin A, FK506 and rapamycin. Further study has shown that the inhibition of rotamase activity, in and of itself, does not result in immunosuppressant activity. Schreiber et al., Science, 1990, vol. 250, pp. 556-559. Instead, immunosuppression appears to stem from the formulation of a complex of immunosuppressant drug and immunophilin. It has been shown that immunophilin-drug complexes interact with ternary protein targets as their mode of action. Schreiber et al., Cell, 1991, vol. 66, pp. 807-815. In the case of FKBP-FK506 and cyclophilin-CsA, the immunophilin-drug complexes bind to the enzyme calcineurin and inhibit the T-cell receptor signaling which leads to T-cell proliferation. Similarly, the immunophilin-drug complex of FKBP-rapamycin interacts with the RAFT1/FRAP protein and inhibits the IL-2 receptor signaling.

Immunophilins have been found to be present at high concentrations in the central nervous system. Immunophilins are enriched 10-50 times more in the central nervous system than in the immune system. Within neural tissues, immunophilins appear to influence nitric oxide synthesis, neurotransmitter release and neuronal process extension.

Surprisingly, it has been found that certain low molecular weight, small peptidic sequences with a high affinity for cyclophilin A are potent rotamase inhibitors and exhibit excellent neurotrophic effects. These findings suggest the use of cyclophilin rotamase inhibitors in treating various peripheral neuropathies and enhancing neuronal regrowth in the central nervous system (CNS). Studies have demonstrated that neurodegenerative disorders such as Alzheimer's disease, Parkinson's disease, and amyotrophic lateral sclerosis (ALS) may occur due to the loss, or decreased availability, of a neurotrophic substance specific for a particular population of neurons affected in the disorder.

Several neurotrophic factors affecting specific neuronal populations in the central nervous system have been identified. For example, it has been hypothesized that Alzheimer's disease results from a decrease or loss of nerve growth factor (NGF). It has thus been proposed to treat SDAT patients with exogenous nerve growth factor or other neurotrophic proteins, such as brain derived growth factor, glial derived growth factor, ciliary neurotrophic factor and neurotropin-3, to increase the survival of degenerating neuronal populations.

Clinical application of these proteins in various neurological disease states is hampered by difficulties in the delivery and bioavailability of large proteins to nervous system targets. By contrast, immunosuppressant drugs with neurotrophic activity are relatively small and display excellent bioavailability and specificity. However, when administered chronically, immunosuppressant drugs exhibit a number of potentially serious side effects including nephrotoxicity, such as impairment of glomerular filtration and irreversible interstitial fibrosis (Kopp et al., J. Am. Soc. Nephrol., 1991, 1:162); neurological deficits, such as involuntary tremors, or non-specific cerebral angina, such as non-localized headaches (de Groen et al., N. Engl. J. Med., 1987, 317:861); and vascular hypertension with complications resulting therefrom (Kahan et al., N. Engl. J. Med., 1989, 321:1725).

In order to prevent the side effects associated with the use of the immunosuppressant compounds, the present invention provides a method of effecting a neuronal activity in an animal, which comprises: administering to the animal an effective amount of a non-immunosuppressive neurotrophic compound having an affinity for a cyclophilin-type immunophilin, wherein the immunophilin exhibits rotamase activity and the neurotrophic compound inhibits the rotamase activity of the immunophilin.

Such a method promotes and regenerates neuronal growth in various neuropathological situations where neuronal repair can be facilitated, including: peripheral nerve damage caused by physical injury or disease state such as diabetes; physical damage to the central nervous system (spinal cord and brain); brain damage associated with stroke; and neurological disorders relating to neurodegeneration, such as Parkinson's disease, SDAT (Alzheimer's disease), and amyotrophic lateral sclerosis.

The present invention further relates to neurotrophic low molecular weight, small molecule cyclophilin inhibitor compounds having an affinity for cyclophilin-type immunophilins. Once bound to these proteins, the neurotrophic compounds are potent inhibitors of the enzyme activity associated with immunophilin proteins, particularly peptidyl-prolyl isomerase, or rotamase, enzyme activity. A key feature of the compounds of the present invention is that they do not exert any significant immunosuppressive activity in addition to their neurotrophic activity.

Exemplary of the compounds of this invention are the following compounds:

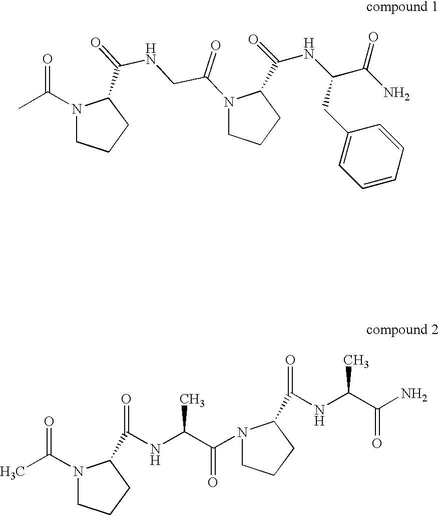

In a preferred embodiment, the neuronal activity is treatment of a neurological disorder selected from the group consisting of peripheral neuropathy caused by physical injury or disease state, physical damage to the brain, physical damage to the spinal cord, stroke associated with brain damage, and neurological disorder relating to neurodegeneration.

In a most preferred embodiment, the neuronal activity is treatment of a neurological disorder relating to neurodegeneration, said disorder selected from the group consisting of Alzheimer's disease, Parkinson's disease, and amyotrophic lateral sclerosis.

### BRIEF DESCRIPTION OF THE DRAWINGS

FIG. 1 shows the promotion of neurite outgrowth in chick sensory neurons by compound 1 at 1 mM.

FIG. 2 shows the promotion of neurite outgrowth in chick sensory neurons by compound 2 at 1 mM.

### DETAILED DESCRIPTION OF THE INVENTION

### Definitions

“Alkyl” means a branched or unbranched saturated hydrocarbon chain containing 1 to 6 carbon atoms, such as methyl, ethyl, propyl, iso-propyl, butyl, iso-butyl, tert-butyl, n-pentyl, n-hexyl, and the like, unless otherwise indicated.

“Alkoxy” means the group —OR wherein R is alkyl as herein defined. Preferably, R is a branched or unbranched saturated hydrocarbon chain containing 1 to 3 carbon atoms.

“Halo” means fluoro, chloro, bromo, or iodo, unless otherwise indicated.

“Phenyl” includes all possible isomeric phenyl radicals, optionally monosubstituted or multi-substituted with substituents selected from the group consisting of alkyl, alkoxy, hydroxy, halo, and haloalkyl.

“Treatment” covers any treatment of a disease and/or condition in an animal, particularly a human, and includes:

(i) preventing a disease and/or condition from occurring in a subject which may be predisposed to the disease and/or condition but has not yet been diagnosed as having it;

(ii) inhibiting the disease and/or condition, i.e., arresting its development; and

(iii) relieving the disease and/or condition i.e., causing regression of the disease and/or condition.

The neurotrophic low molecular weight, small molecule peptidic cyclophilin inhibitor compounds of the present invention have an affinity for cyclosporin A binding proteins such as cyclophilin A. When the neurotrophic compounds are bound to cyclophilin, they have been found to inhibit the prolyl-peptidyl cis-trans isomerase activity, or rotamase, activity of the binding protein and unexpectedly stimulate neurite growth.

The compounds of the present invention may be synthesized according to any procedure known in the art. For illustration, Example I, below, sets forth a representative procedure for synthesizing inventive compounds useful in the methods of this invention.

### Method of Use

The compounds of the present invention have an affinity for cyclosporin-type binding proteins, particularly cyclophilin A, which is present in the brain. When the compounds bind to cyclophilin A in the brain, they exhibit excellent neurotrophic activity. This activity is useful in the stimulation of damaged neurons, the promotion of neuronal regeneration, the prevention of neurodegeneration, and the treatment of several neurological disorders know to be associated with neuronal degeneration and peripheral neuropathies.

For the foregoing reasons, the present invention further relates to a method of effecting a neuronal activity in an animal, comprising:

administering to the animal an effective amount of a neurotrophic compound having an affinity for a cyclophilin-type immunophilin, wherein the immunophilin exhibits rotamase activity and the neurotrophic compound inhibits the rotamase activity of the immunophilin.

In a preferred embodiment, the neuronal activity is selected from the group consisting of stimulation of damaged neurons, promotion of neuronal regeneration, prevention of neurodegeneration and treatment of neurological disorder.

The neurological disorders that may be treated include but are not limited to: trigeminal neuralgia; glossopharyngeal neuralgia; Bell's Palsy; myasthenia gravis; muscular dystrophy; amyotrophic lateral sclerosis; progressive muscular atrophy; progressive bulbar inherited muscular atrophy; herniated, ruptured or prolapsed invertebrate disk syndromes; cervical spondylosis; plexus disorders; thoracic outlet destruction syndromes; peripheral neuropathies such as those caused by lead, dapsone, ticks, porphyria, or Guillain-Barré syndrome; Alzheimer's disease; and Parkinson's disease.

The compounds of the present invention are particularly useful for treating a neurological disorder selected from the group consisting of: peripheral neuropathy caused by physical injury or disease state, physical damage to the brain, physical damage to the spinal cord, stroke associated with brain damage, and neurological disorder relating to neurodegeneration. Examples of neurological disorders relating to neurodegeneration are Alzheimer's disease, Parkinson's disease, and amyotrophic lateral sclerosis.

For these purposes, the compounds may be administered orally, parenterally, by inhalation spray, topically, rectally, nasally, buccally, vaginally or via an implanted reservoir in dosage formulations containing conventional non-toxic pharmaceutically-acceptable carriers, adjuvants and vehicles. The term parenteral as used herein includes subcutaneous, intravenous, intramuscular, intraperitoneally, intrathecally, intraventricularly, intrasternal and intracranial injection or infusion techniques.

To be effective therapeutically on a central nervous system target, the compounds should readily penetrate the blood-brain barrier when peripherally administered. Compounds which cannot penetrate the blood-brain barrier can be effectively administered by an intraventricular route.

The compounds may be administered in the form of sterile injectable preparations, for example, as sterile injectable aqueous or oleaginous suspensions. These suspensions may be formulated according to techniques know in the art using suitable dispersing or wetting agents and suspending agents. The sterile injectable reparations may also be sterile injectable solutions or suspensions in non-toxic parenterally-acceptable diluents or solvents, for example, as solutions in 1,3-butanediol. Among the acceptable vehicles and solvents that may be employed are water, Ringer's solution and isotonic sodium chloride solution. In addition, sterile, fixed oils are conventionally employed as solvents or suspending mediums. For this purpose, any bland fixed oil such as a synthetic mono- or di-glyceride may be employed. Fatty acids such as oleic acid and its glyceride derivatives, including olive oil and castor oil, especially in their polyoxyethylated versions, are useful in the preparation of injectables. These oil solutions or suspensions may also contain long-chain alcohol diluents or dispersants.

Additionally, the compounds may be administered orally in the form of capsules, tablets, aqueous suspensions or solutions. Tablets may contain carriers such as lactose and cornstarch, and/or lubricating agents such as magnesium stearate. Capsules may contain diluents including lactose and dried cornstarch. Aqueous suspensions may contain emulsifying and suspending agents combined with the active ingredient. The oral dosage forms may further contain sweetening and/or flavoring and/or coloring agents.

The compounds may also be administered rectally in the form of suppositories. These compositions can be prepared by mixing the drug with a suitable non-irritating excipient which is solid at room temperature, but liquid at rectal temperature and, therefore, will melt in the rectum to release the drug. Such materials include cocoa butter, beeswax and polyethylene glycols.

Furthermore, the compounds may be administered topically, especially when the conditions addressed for treatment involve areas or organs readily accessible by topical application, including neurological disorders of the eye, the skin, or the lower intestinal tract. Suitable topical formulations can be readily prepared for each of these areas.

For topical application to the eye, or ophthalmic use, the compounds can be formulated as micronized suspensions in isotonic, pH adjusted sterile saline, or, preferably, as a solution in isotonic, pH adjusted sterile saline, either with or without a preservative such as benzylalkonium chloride. Alternatively, the compounds may be formulated into ointments, such as petrolatum, for ophthalmic use.

For topical application to the skin, the compounds can be formulated into suitable ointments containing the compounds suspended or dissolved in, for example, mixtures with one or more of the following: mineral oil, liquid petrolatum, white petrolatum, propylene glycol, polyoxyethylene polyoxypropylene compound, emulsifying was and water. Alternatively, the compounds can be formulated into suitable lotions or creams containing the active compound suspended or dissolved in, for example, a mixture of one or more of the following: mineral oil, sorbitan monostearate, polysorbate 60, cetyl ester was, cetearyl alcohol, 2-octyldodecanol, benzyl alcohol and water.

Topical application to the lower intestinal tract can be effected in a rectal suppository formulations (see above) or in suitable enema formulations.

Dosage levels on the order of about 0.1 mg to about 10,000 mg of the active ingredient compound are useful in the treatment of the above conditions, with preferred levels of about 0.1 mg to about 1,000 mg. The amount of active ingredient that may be combined with the carrier materials to produce a single dosage form will vary depending upon the host treated and the particular mode of administration.

It is understood, however, that a specific dose level for any particular patient will depend upon a variety of factors, including the activity of the specific compound employed; the age, body weight, general health, sex and diet of the patient; the time of administration; the rate of excretion; drug combination; the severity of the particular disease being treated; and the form of administration.

The compounds can be administered with other neurotrophic agents such as nerve growth factor (NGF), glial cell line-derived neurotrophic factor, brain derived neurotrophic factor, ciliary neurotrophic factor, and neurotrophin-3. The dosage level of other neurotrophic drugs will depend upon the factors previously stated and the neurotrophic effectiveness of the drug combination.

### EXAMPLES

The following examples are illustrative of the present invention and are not intended to be limitation thereon.

### Example I

### Synthesis of Compounds

Exemplified for compound 1: Rink resin 0.25 g (0.44 meq/g) was transferred to a reactor column and washed with dimethyl formamide (DMF) (3×5 min) followed by 50% piperidine in DMF (2×10 min) to remove protecting group 9-fluorenylmethoxycarbonyl (Fmoc). The resin was washed with DMF (5×5 min) and a first amino acid was added. The first protected amino acid Fmoc-Phe (0.25 mmol) was dissolved together with 1-hydroxybenzotriazole (HOBt; 0.25 mmol) in 2.5 ml DMF for pre-activation (3 min) followed by benzotriazolyloxy (tris) dimethylamino-phosphonium hexafluorophosphate (BOP; 0.25 mmole) and 4-methylmorpholine (NMM; 0.375 mmole). The mixture was immediately poured into the reactor column and shaken for 2 hours. After 3×5 min DMF washing, negative color test for residual amine was obtained. The DMF wash was repeated and the subsequent residues (Fmoc-Pro, Fmoc-Gly, Fmoc-Pro) were added using the same deprotection, washing, coupling, washing cycle until all designed amino acids for the sequence had been connected. After the last amino acid was coupled, the Fmoc was removed by 50% piperidine in DMF (2×10 min) as before followed by DMG washing (5×5) min. The resin was acetylated of terminal amino group with 2 ml mixture of DMF: acetic anhydride: N-ethyldiisopropylamine=193:6:1 (v/v/v) for 90 minutes at room temperature. The final peptide resin was washed with DMF (3×5 min), t-amyl alcohol (2×3 min), acetic acid (2×3 min), t-amyl alcohol (2×3 min), ether (3×3 min), and dried in high vacuum overnight. The dried peptide resin was treated with 2 ml TFA:Phenol:H₂O=90:5:5 for 2 hours at room temperature. The resin was filtered, washed thoroughly with TFA and the total filtrate evaporated under N₂. Methyl t-butyl ether (50 mL) was added to the residue and the resultant white precipitate was collected after centrifugation. The NMR spectrum was consistent with the expected structure.

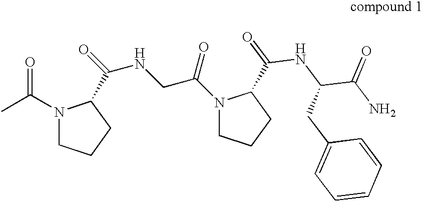

The following compound 2 was prepared according to the same method, except that Fmoc-Ala was used as a first protected amino acid, and Fmoc-Pro, Fmoc-Ala, and Fmoc-Pro were subsequently added using the same deprotection-washing-coupling-washing-cycle described above.

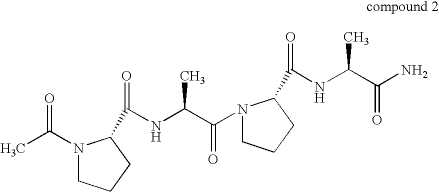

### Example II

### Ki Test Procedure

Inhibition of the peptidyl-prolyl isomerase (rotamase) activity of the inventive compounds can be evaluated by known methods described in the literature (Harding, et al., Nature, 1989, 341:758-760; Holt et al. J. Am. Chem. Soc., 115:9923-9938). These values are obtained as apparent Ki's. The cis-trans isomerization of an alanine-proline bond in a model substrate, N-succinyl-Ala-Ala-Pro-Phe-p-nitroanilide, is monitored spectrophotometricallly in a chymotrypsin-coupled assay, which releases para-nitroanilide from the trans for of the substrate. The inhibition of this reaction caused by the addition of different concentrations of inhibitor is determined, and the data is analyzed as a change in first-order rate constant as a function of inhibitor concentration to yield the apparent Ki values. The absorbance at 390 nm versus time is monitored for 90 seconds using a spectrophotometer and the rate constants are determined from the absorbance versus time data files.

### Example III

### Chick Dorsal Root Ganglion Cultures and Neurite Outgrowth

The neurotrophic effects of the cyclophilin inhibitors were demonstrated by evaluating the ability of the compounds to promote neurite outgrowth in cultured chick sensory neurons form dorsal root ganglia. Dorsal root ganglia were dissected from chick embryos of ten-day gestation. Whole ganglion explants were cultured on thin layer Matrigel-coated 12 well plates with Liebovitz L15 plus high glucose media supplemented with 2 mM glutamine and 10% fetal calf serum, and also containing 10 μM cytosine 3-D arabinofuranoside (Ara C) at 37° C. in an environment containing 5% CO₂. Twenty-four hours later, the DRGs were treated with various concentrations of nerve growth factor, immunophilin ligands or combinations of NFG plus drugs. Forty-eight hours after drug treatment, the ganglia were visualized under phase contrast or Hoffman Modulation contrast with a Zeiss Axiovert inverted microscope. Photomicrographs of the explants were made, and neurite outgrowth was quantitated. Neurites longer that the DRG diameter were counted as positive, with total number of neurites quantitated per each experimental condition. Three to four DRGs are cultured per well, and each treatment was performed in duplicate.

The maximal increase in the number of processes, their length and branching is quite similar at maximally effective contractions of the cyclophilin ligands and of NGF (100 ng/ml). With progressively increasing concentrations of the various drugs, one observes a larger number of processes, more extensive branching and a grater length of individual processes.

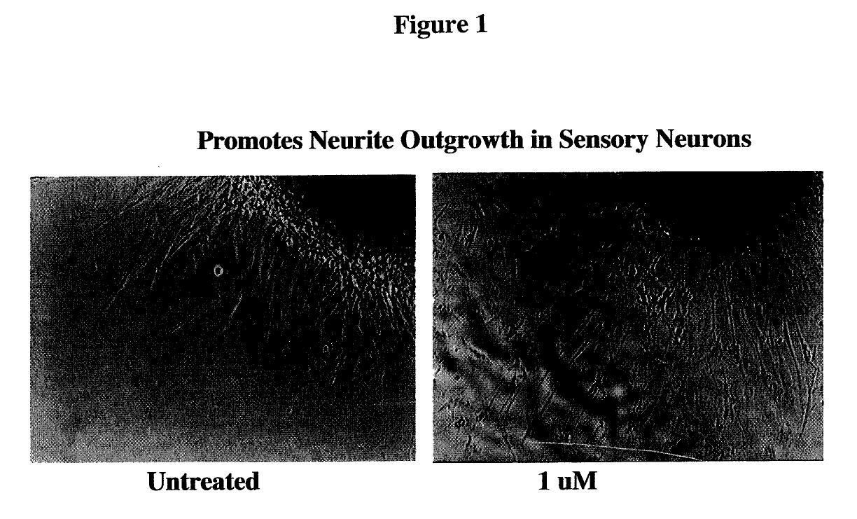

FIG. 1 shows the action of compound 1 on chick sensory neurons; at 1 mM concentration, the compounds exert powerful neurotrophic effects, as seen by the eliciting of long fibers form the cell body.

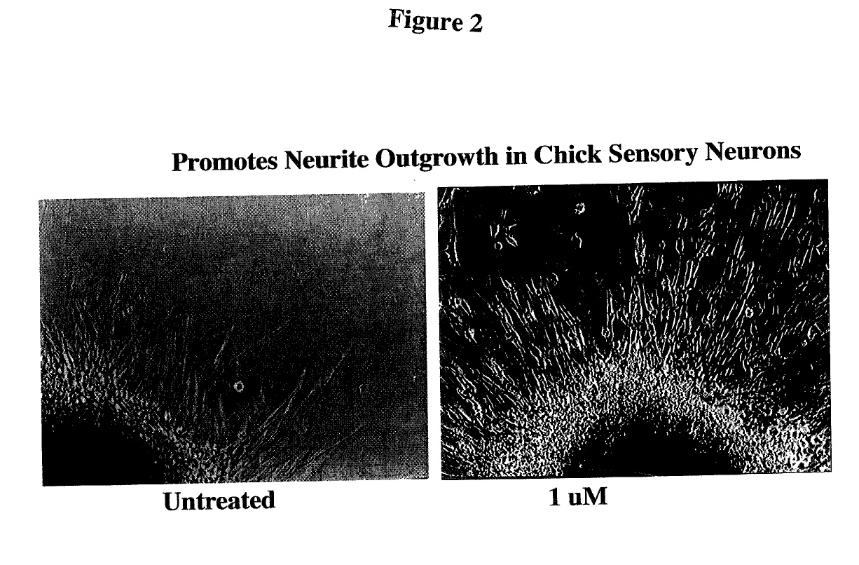

Similarly, FIG. 2 shows the potent neurotrophic effects of compound 2 on these neuronal cultures.

The invention being thus described, it will be obvious that the same may be varied in many ways. Such variations are not to be regarded as a departure from the spirit and scope of the invention and all such modifications are intended to be included within the scope of the following claims.

We claim:

1. A method of effecting a neuronal activity in an animal, comprising:
administering to the animal an effective amount of a non-immunosuppressive neurotrophic compound having an affinity for a cyclophilin-type immunophilin, wherein the immunophilin exhibits rotamase activity and the neurotrophic compound inhibits the rotamase activity of the immunophilin.

2. The method according to claim 1, wherein the neuronal activity is selected from the group consisting of stimulation of damaged neurons, promotion of neuronal regeneration, prevention of neurodegeneration and treatment of neurological disorder.

3. The method according to claim 2, wherein the neurological disorder is selected from the group consisting of peripheral neuropathy caused by physical injury or disease state, physical damage to the brain, physical damage to the spinal cord, stroke associated with brain damage, and neurological disorder relating to neurodegeneration.

4. The method according to claim 3, wherein the neurological disorder relating to neurodegeneration is selected from the group consisting of Alzheimer's disease, Parkinson's disease, and amyotrophic lateral sclerosis.

5. The method according to claim 3, wherein the neurological disorder relating to neurodegeneration is Alzheimer's disease.

6. The method according to claim 3, wherein the neurological disorder relating to neurodegeneration is Parkinson's disease

7. The method according to claim 3, wherein the neurological disorder relating to neurodegeneration is amyotrophic lateral sclerosis.

8. The method according to claim 3, wherein the neurological disorder is peripheral neuropathy caused by physical injury or disease state.

9. The method according to claim 8, wherein the disease state is diabetes.

10. The method according to claim 3, wherein the neurological disorder is stroke associated with brain damage.

11. The method of claim 1, wherein the cyclophilin-type immunophilin is cyclophilin A.

12. The method of claim 11, wherein the neuronal activity is selected from the group consisting of stimulation of damaged neurons, promotion of neuronal regeneration, prevention of neurodegeneration and treatment of neurological disorder.

13. The method of claim 12, wherein the neurological disorder is selected from the group consisting of peripheral neuropathy caused by physical injury or disease state, physical damage to the brain, physical damage to the spinal cord, stroke associated with brain damage, and neurological disorder relating to neurodegeneration.

14. The method of claim 13, wherein the neurological disorder relating to neurodegeneration is selected from the group consisting of Alzheimer's disease, Parkinson's disease, and amyotrophic lateral sclerosis.

15. The method of claim 1, wherein the neurotrophic compound is capable of penetrating the blood-brain barrier following peripheral administration.

16. The method of claim 15, wherein the cyclophilin-type immunophilin is cyclophilin A.
